(19) United States
(12) Patent Application Publication
(10) Pub. No.: US 2025/0041466 A1
(43) Pub. Date: Feb. 6, 2025

(54) DISINFECTION DEVICE, ARRANGEMENT AND OPERATION METHOD

(71) Applicant: ams-OSRAM International GmbH, Regensburg (DE)

(72) Inventors: Ruth BOSS, Neutraubling (DE); Dennis HAN, Seoul (KR)

(21) Appl. No.: 18/709,391

(22) PCT Filed: Nov. 3, 2022
(86) PCT No.: PCT/EP2022/080634

§ 371 Date: May 10, 2024

(30) Foreign Application Priority Data
Nov. 12, 2021 (DE) 10 2021 129 558.0

(51) Int. Cl. A61L 2/10; A61L 2/24; A61L 2/26; F21V 9/32

(52) U.S. Cl. CPC A61L 2/10; A61L 2/24; A61L 2/26; F21V 9/32; A61L 2202/11; A61L 2202/121; A61L 2202/122; A61L 2202/123; A61L 2202/14

(57) ABSTRACT

In at least one embodiment, the disinfection device comprises: —a housing body, —a semiconductor chip located in the housing body and configured to emit ultraviolet C radiation (UVC), and—a security sensor located in the housing body, wherein—the housing body forms a radiation shield preventing the ultraviolet C radiation (UVC) from leaving the disinfection device, —the housing body comprises an opening configured for actuating a button by a user, to actuate the button the user has to grasp into the opening, —the semiconductor chip is configured to irradiate at least part of the opening and/or the button, and—the disinfection device is configured as an attachment device to be disposed over the button.

A disinfection device is provided. A method for operating such a disinfection device is also provided.

Documents CN 111 228 553 A and CN 202 808 105 U refer to disinfection devices.

A problem to be solved is to provide a disinfection device that can be operated efficiently and safely.

This object is achieved, inter alia, by a disinfection device, by a disinfection arrangement and by an operation method as defined in the independent patent claims. Exemplary further developments constitute the subject-matter of the dependent claims.

For example, the disinfection device and/or the disinfection arrangement is an elevator floor selection module or an opener in a vehicle, like a car or airplane or ship, with self-disinfection and protection, wherein disinfection is achieved by a UVC-emitting LED chip which is close to the surface to be disinfected and, thus, needs to be turned on only in short time intervals, and only a comparably low optical output power is needed.

According to at least one embodiment, the disinfection device comprises a housing. The housing may be the part of the disinfection device that mechanically carries and protects the other components of the disinfection device. The housing includes a housing body which is, for example, of a plastic material.

According to at least one embodiment, the disinfection device comprises one or a plurality of semiconductor chips configured to emit ultraviolet C radiation, UVC for short. UVC refers to the spectral range from 100 nm to 280 nm. The at least one semiconductor chip is preferably located within the housing body. For example, the at least one semiconductor chip is based on the semiconductor material AlGaN and may have a peak emission wavelength of at least 260 nm and/or of at most 280 nm.

According to at least one embodiment, the disinfection device comprises one or a plurality of security sensor. The at least one security sensor is located in or at the housing body. For example, the at least one security sensor is a light barrier which may be based on visible or near-infrared radiation.

According to at least one embodiment, the housing body forms and/or comprises a radiation shield. By means of the radiation shield, the UVC is prevented from leaving the disinfection device. This means, for example, that at most 10% or at most 2% or at most 0.2% of the UVC generated by the at least one semiconductor chip can leave the disinfection device in its intended use. This is achieved in particular by means of the shape and UVC reflecting and transmitting properties of the radiation shield.

According to at least one embodiment, the housing body comprises an opening configured for actuating a button by a user. Per button, there can be exactly one such opening. The user can thus actuate the button by grasping into and/or through the opening. It is possible that the opening runs completely through the disinfection device from a top side of the housing to the button, or that there is at least one intermediate component between the top side and the button. Thus, the button may directly be actuated by the user, or the user may actuate the intermediate component which in turn actuates the button.

For example, the button is an elevator button or a button in another transport means like a bus or train or subway. For example, the button is located in a health-sensitive area like a hospital or a nursing home. The button may be accessible by a large number of different people like visitors, patients, employees or passengers.

According to at least one embodiment, the at least one semiconductor chip is configured to irradiate at least part of the opening and/or of the button with the UVC. For example, the at least one semiconductor chip directly or indirectly irradiates the button and/or irradiates the intermediate component. Preferably, all surfaces of the disinfection device and/or of the button the user touches in the intended use of the disinfection device and/or of the button are irradiated with the UVC.

According to at least one embodiment, the disinfection device is configured as an attachment device to be disposed over the button. That is, the disinfection device can be a supplement part to be arranged on an existing button, in particular without the need to modify the button or a board the button is arranged on.

In at least one embodiment, the disinfection device comprises:
a housing body,
a semiconductor chip located in the housing body and configured to emit ultraviolet C radiation, and
a security sensor located in the housing body, wherein
the housing body forms a radiation shield preventing the ultraviolet C radiation from leaving the disinfection device,
the housing body comprises an opening configured for actuating a button by a user, to actuate the button the user has to grasp into the opening,
the semiconductor chip is configured to irradiate at least part of the opening and/or the button, and
the disinfection device is configured as an attachment device to be disposed over the button. Optionally, one of the following possibilities holds true:
the housing further comprises an intermediate plate located movably in the opening between a top side of the housing body and the button, the intermediate plate is configured to press the button when being actuated by the user, or
the housing further comprises a shutter forming part of the radiation shield, the shutter closes the opening when being closed and allows for accessing the button when being opened, the security sensor is configured to provide an opening signal for the shutter when the semiconductor chip is turned off and when the user approaches the shutter.

With this disinfection device, effective disinfection of all kinds of buttons used in elevators/transports/hospitals touched by lots of people can be achieved. Further, the button can be used in vehicles, like cars, ships or airplanes, for example, in rental cars or as an airplane luggage door opener. Because the disinfection device includes the security sensor, exposure of human skin to the UVC from the semiconductor chip can be prevented.

Some technical problems can be solved all together with the disinfection device described herein:
by using UVC, harmful pathogens can be killed or disabled;
energy and space can be saved by the compact design of the disinfection device;
UVC radiation can efficiently and effectively be prevented from exposure to humans;
the disinfection device can be an add-on device which allows for easy installation and replacement.

A primary functionality of the disinfection device described herein is to kill or disable pathogens, such as SARS-Cov-2 or many other viruses that cause diseases to human being, for example, at floor selection buttons or any kind of door openers which are frequently touched with fingers.

In the disinfection device described herein, disinfection is more focused on surfaces that are frequently touched by people rather than everything around, which makes disinfection more energy and space efficient using compact LEDs instead of long UVC-emitting linear tubes or embedding UVC light sources into luminaires on a ceiling. Hence, less radiation flux and dramatically less exposure time will be required than in conventional designs. UVC can be prevented from reaching human skin or eye using sensors and having the UVC-emitting semiconductor chip within the compact housing body. The disinfection device can be easily installed or replaced for new ones at various types of floor selection buttons or even other buttons like door openers.

According to at least one embodiment, the opening runs completely through the housing body. Hence, seen in top view, the button can be exposed by means of the opening and the button which is no part of the disinfection device may not be covered by the disinfection device. Thus, the opening is configured to allow the user to freely access the button.

According to at least one embodiment, seen in top view of the housing body, the opening is completely surrounded by the housing body. For example, the opening is located in a center of the housing body. The opening may be in a center point of the housing body, and the opening and/or the housing body may be of point symmetry or rotational symmetry.

According to at least one embodiment, the semiconductor chip is located in a cavern within the housing body. For example, the cavern is formed around or all around the opening, seen in top view of the housing body. The cavern may directly start at the opening. The opening and the cavern may be arranged in a disjunctive manner.

According to at least one embodiment, a top face of the cavern remote from the button is partially or completely coated with a reflection layer for the UVC. The reflection layer may be part of the radiation shield. For example, a reflectivity of the reflection layer for the UVC emitted by the at least one semiconductor chips is at least 75% or at least 85%. It is possible that the reflection layer is limited to the top face or to parts of the top face.

According to at least one embodiment, side faces of the opening are coated with an absorption layer for the UVC. The absorption layer can be part of the radiation shield. For example, an absorption coefficient of the absorption layer for the UVC emitted by the at least one semiconductor chips is at least 75% or at least 85%. For example, a transmission coefficient of the absorption layer for the UVC emitted by the at least one semiconductor chips is at most 15% or at most 3%.

According to at least one embodiment, the housing further comprises an intermediate plate located movably in the opening. For example, the intermediate plate is located between the exterior top side of the housing body and the button. The intermediate plate may be configured to press the button when being actuated by the user. Hence, the button may not be directly pressed by the user but only indirectly via the intermediate plate. Thus, it may be possible to avoid that the button is exposed to the UVC and destruction of the button due to UVC can be avoided.

According to at least one embodiment, the intermediate plate is fully or partially transparent for the UVC emitted by the at least one semiconductor chip. ‘Fully’ may mean that a transmission coefficient for the UVC is at least 85%. For example, the transmission coefficient of the intermediate plate for the UVC is at least 25% or at least 40% or at least 60%. Thus, a top surface of the intermediate plate remote from the button can be disinfected with the UVC although the UVC needs to travel through the intermediate plate.

According to at least one embodiment, the intermediate plate is located between the top side and the semiconductor chip. Thus, the at least one semiconductor chip can be protected from moisture or dust by means of the intermediate plate and the cavern may be a closed volume because of the intermediate plate.

According to at least one embodiment, the housing further comprises one or a plurality of shutters. The at least one shutter is part of the radiation shield. If there is a plurality of shutters, there may be a one-to-one assignment between the shutters and the buttons.

According to at least one embodiment, the shutter closes the opening when being closed and allows for accessing the button when being opened. In particular, the shutter is closed when UVC is emitted by the at least one semiconductor chip.

According to at least one embodiment, the security sensor is configured to provide an opening signal for the shutter when the semiconductor chip is turned off and/or when the user approaches the shutter. It is possible that the shutter is closed only during the short time interval when the UVC radiation is emitted by the at least one semiconductor chip so that a standard position of the shutter may be the open state. Otherwise, the standard position of the shutter may be the closed state so that the shutter is open only when a user approaches the button, indicated in particular by means of the security sensor.

According to at least one embodiment, the shutter is located at a bottom of a forecourt of the opening, the forecourt is surrounded all around by a funnel of the radiation shield, seen in a lateral direction. In other words, there can be a recess at the top side of the housing, and said recess corresponds to the forecourt. When the shutter is closed, the shutter separates the forecourt from the rest of the opening.

The funnel can be of a UVC absorbing material and/or can be opaque to the UVC emitted by the at least one semiconductor chip.

According to at least one embodiment, the housing further comprises a joint like a tilt joint. A first end at the tilt joint is configured to be pulled by the user, and a second end at the tilt joint is configured to push the button. Thus, the first end may be regarded as a hand lever handled by the user and the second end may be regarded as a button lever that presses the button. If the tilt joint is present, the button may completely be covered by the disinfection device. The tilt joint may alternatively be a pivot joint or an articulated joint or the like.

If there is a plurality of the buttons assigned to the disinfection device, then there can be a one-to-one assignment between the shutters, funnels and/or tilt joints.

According to at least one embodiment, the at least one semiconductor chip is configured to irradiate an interior side of the second end, in particular only an interior side of the second end. Said interior side is part of the opening.

According to at least one embodiment, the opening is located between the at least one semiconductor chip and the second end. Hence, the user grasps into the space between the at least one semiconductor chip and the second end and pulls the second end to actuate the respective button.

According to at least one embodiment, the disinfection device further comprises one or a plurality of indicator light sources. The at least one indicator light source is configured to indicate when the at least one semiconductor chip is turned on and/or when the at least one semiconductor chip is turned off.

For example, the indicator light uses one or a plurality of colors to indicate operation and/or non-operation of the at least one semiconductor chip. As an option, the indicator light uses blue and purple, or red and green, as indicator colors. For example, the indicator light source comprises at least one green emitting LED chip and at least one red emitting LED chip, where LED means light-emitting diode, and/or comprises a blue emitting LED chip and a red or purple emitting LED chip.

According to at least one embodiment, the disinfection device further comprises one or a plurality of phosphor sheets, also referred to as converter plates. The at least one phosphor sheet is configured to indicate when the at least one semiconductor chip is turned on and/or when the at least one semiconductor chip is turned off by converting the UVC radiation from the at least one semiconductor chip into visible light. The at least one phosphor sheet is embedded in the housing body, for example.

According to at least one embodiment, the indicator light source is embedded in the housing body and the housing body is of a material transparent for visible light. Thus, the housing body can be configured as a light guide for indicator light emitted by the indicator light source.

According to at least one embodiment, the disinfection device further comprises a battery, the disinfection device is configured to be powered by the battery. Thus, the disinfection device can be applied on the button without the need of any further electrification. Alternatively or additionally to a battery, the disinfection device may include a photovoltaic element and/or may be connected with an external power line, like a low-voltage direct current power line which may also supply the button with current, for example.

According to at least one embodiment, a distance between a surface to be disinfected by the UVC and the at least one semiconductor chip is at most 4 cm or is at most 2 cm. Thus, even if the at least one semiconductor chip has a comparably low optical output power concerning the UVC of, for example, a couple of mW, then a relative high luminous flux of the UVC per unit area can be achieved because of said short distance.

According to at least one embodiment, the security sensor comprises one or a plurality of light barriers. The at least one light barrier is configured to check an entrance of the opening, for example. Hence, it can be avoided that the at least one semiconductor chips is in operation when a user approaches the button and/or grasps into the opening. Hence, exposure of the user to the UVC can be avoided.

According to at least one embodiment, the at least one semiconductor chip is a light-emitting diode chip or a laser diode chip.

According to at least one embodiment, the button is a push-button, a pressure key, a switch, a shifter, an opener, a knob, a lever, a touchpad, a shutter, a shutter membrane, a cover film or a handgrip. For example, the button is an elevator push-button or a car opener or a luggage door opener or a control knob, or may be the touchpad of a console or a computer or a user equipment, like a mobile phone. In each case, the button is subject to touch action by a user. Hence, the term ‘button’ is not necessarily limiting to push-buttons. Terms for the ‘pushing’ of the button include pressing, depressing, mashing, slapping, hitting, and punching, for example.

A disinfection arrangement is additionally provided. The disinfection arrangement comprises one or a plurality of the disinfection devices as indicated in connection with at least one of the above-stated embodiments. Features of the disinfection arrangement are therefore also disclosed for the disinfection device and vice versa.

In at least one embodiment, the disinfection arrangement comprises at least one disinfection device and at least one button. The disinfection device is attached over the assigned button so that said button can be actuated only by means of grasping, by the user, into the opening.

According to at least one embodiment, the disinfection device comprises fastener, and the disinfection device is attached over the button by means of the fastener. For example, the fastener comprises an adhesive tape, a clip, a squeeze, a clamp, a screw, a pin, a hook-and-loop and/or a magnet, or the fastener consists of one of these components.

A method for operating the disinfection device is additionally provided. By means of the method, a disinfection device is operated as indicated in connection with at least one of the above-stated embodiments. Features of the disinfection device are therefore also disclosed for the method and vice versa.

In at least one embodiment, the operation method is for operating the disinfection device, wherein during operation the semiconductor chip has an optical output of the ultraviolet C radiation (UVC) of at least 1 mW and of at most 20 mW. Preferably, for disinfection the semiconductor chip is turned on for at least 1 s or at least 10 s and/or for at most 2 min or for at most 20 s.

A disinfection device, a disinfection arrangement and an operation method described herein are explained in greater detail below by way of exemplary embodiments with reference to the drawings. Elements which are the same in the individual figures are indicated with the same reference numerals. The relationships between the elements are not shown to scale, however, but rather individual elements may be shown exaggeratedly large to assist in understanding.

In the figures:

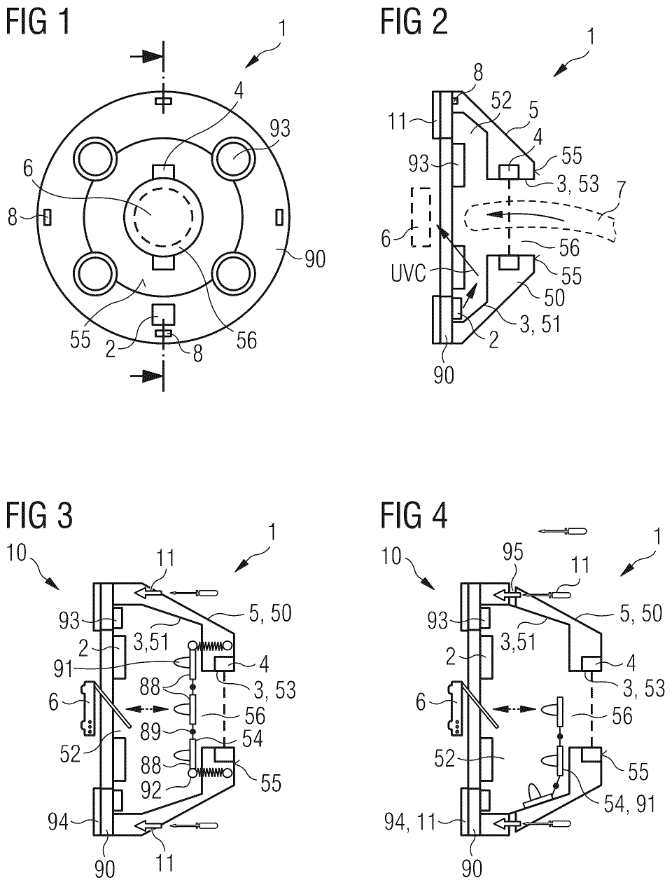

FIG. 1 is a schematic top view of an exemplary embodiment of a disinfection device described herein,

FIG. 2 is a schematic sectional view along the dash-dotted line of the disinfection device of FIG. 1,

FIGS. 3 and 4 are schematic sectional views of exemplary embodiment of disinfection devices and disinfection arrangements described herein,

FIG. 5 is a schematic front view of an exemplary embodiment of a disinfection device and a disinfection arrangement described herein,

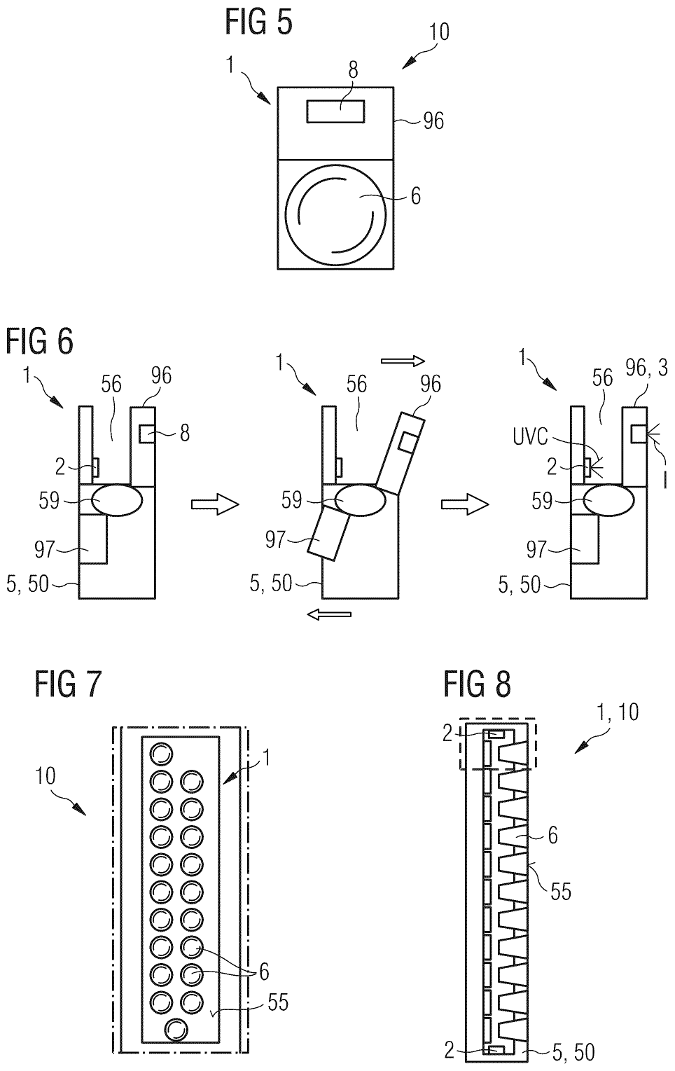

FIG. 6 are schematic sectional views of method steps of an exemplary embodiment of an operation method of the disinfection device of FIG. 5,

FIG. 7 is a schematic top view of an exemplary embodiment of a disinfection arrangement described herein,

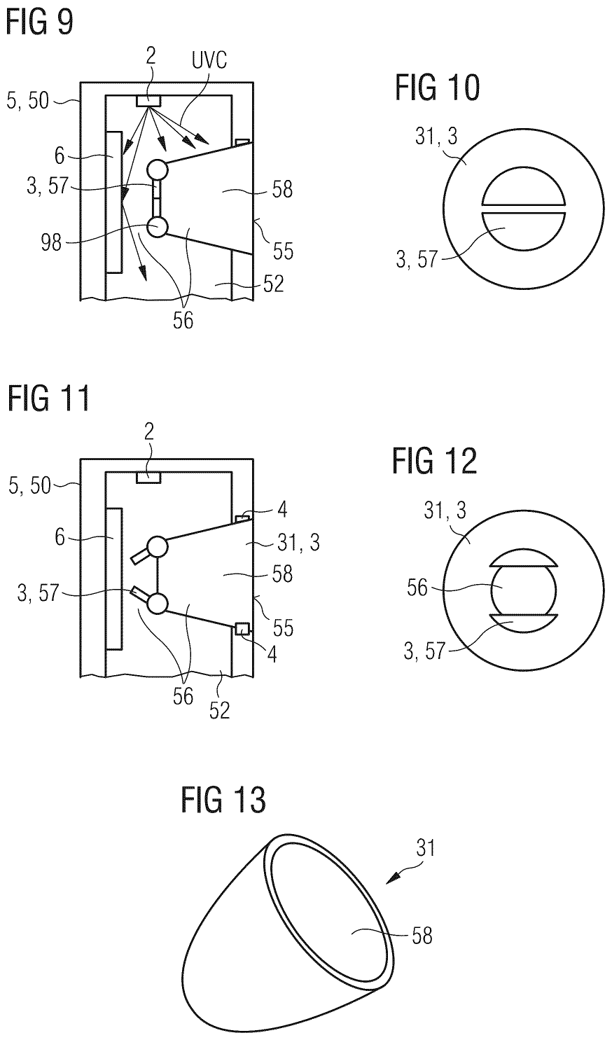

FIGS. 8, 9 and 11 are schematic sectional views of the disinfection arrangement of FIG. 7,

FIGS. 10 and 12 are schematic top views of the disinfection arrangement of FIG. 7, and

FIG. 13 is a schematic perspective view of a funnel of the disinfection arrangement of FIG. 7.

FIGS. 1 and 2 illustrate an exemplary embodiment of a disinfection device 1. The disinfection device 1 comprises a housing 5 in which a UVC emitting semiconductor chip 2 is located. The semiconductor chip 2 is placed in a cavern 52 in a housing body 55 of the housing 5. A peak emission wavelength of the semiconductor chip 2 is, for example, between 260 nm and 280 nm and the semiconductor chip 2 may be based on AlGaN. The disinfection device 1 may be powered by one or a couple of batteries 93 like lithium button cells. The batteries 93 and the semiconductor chip 2 are mounted on a carrier 90 which is, for example, a printed circuit board, PCB for short, comprising FR4, or a metal core board.

An opening 56 runs completely though the housing 5 along a direction perpendicular to a top side 55 of the housing body 50. The opening 56 is configured such that a user 7 can reach with a finger a button 6, the disinfection device 1 is thus located atop the button 6. The button 6 and, of course, the user 7 are not part of the disinfection device 1 and are therefore illustrated by dashed lines. For example, the button 6 is a push-button, a pressure key, a switch, a shifter, an opener, a knob, a lever, a touchpad, a shutter, a shutter membrane, a cover film or a handgrip which is configured to be touched by a user.

For example, a diameter of the opening 56 is at least 1 cm and at most 5 cm. A height of the disinfection device 1 is, for example, at least 5 mm and/or at most 5 cm or at most 2 cm. An overall diameter of the disinfection device 1 is, for example, at least 1.5 times and/or at most 5 times the diameter of the opening 56.

To mount the disinfection device 1 above the button 6, there can be a fastener 11 at a bottom side of the housing 5. For example, the fastener 11 is an adhesive tape or a sticker so that the disinfection device 1 can be applied onto the button 6 without the need of any tools. It is possible that the adhesive tape is removable by means of heat treatment or the like. Preferably, the adhesive tape is protected from being irradiated with the UVC.

In order to avoid emission of the UVC out of the housing 5, a top face of the cavern 52 remote from the bottom side is provided with a reflection layer 51 which is part of a radiation shield 3 for the UVC. For example, the reflection layer 51 is of smoked magnesium oxide or of aluminum. A reflectivity of the reflection layer 51 for the UVC is, for example, between 88% and 93% inclusive.

Thus, the semiconductor chip 2 may have a main emission direction away from the bottom side and towards the reflection layer 51, and the UVC is then distributed by means of the reflection layer 51 across a bottom end of the opening 56 where the button 6 is located. Accordingly, the button 6 can be disinfected by means of the UVC, and the semiconductor chip 2 is located quite close to the bottom end of the opening 56.

Further, to avoid significant leakage of UVC out of the opening 56 towards the top side 55, inner side surfaces of the opening 56 which may run perpendicular to the bottom side may be coated with an absorption layer 53. For example, the absorption layer 53 is based on a zinc oxide paint and may have a reflectivity for the UVC of 5% or less. A length of the inner side surfaces having the absorption layer 53 is, for example, at least once or twice a height of the cavern 52 between the carrier 90 and the reflection layer 51.

For example, in the inner side surfaces of the opening 56 there is a security sensor 4. The security sensor 4 can be a light barrier that checks a top end of the opening 56 remote from the bottom side. When the user 7 grasps into the opening 56, the security sensor 4 hinders or stops operation of the semiconductor chip 2 so that UVC exposure of the user 7 can be avoided. The light barrier may work with near-infrared radiation, that is, with a radiation having a peak wavelength of at least 680 nm and/or of at most 1.5 μm.

As a further option, the disinfection device 1 can include at least one indicator light source 8. For example, the indicator light source 8 is embedded in the housing body 50. Preferably, the housing body 50 is of a material transmissive for visible light and resilient concerning the UVC. For example, the housing body 50 is made of a plastic like PTFE, polytetrafluoroethylene. Thus, the housing body 5 can act as a light guide for indicator light emitted by the indicator light source 8 so that essentially the whole housing body 5 may glare when the indicator light source 8 is turned on. For example, the indicator light source 8 emits red light when the semiconductor chip 2 produces the UVC. As an option, the indicator light source 8 may emit green light when the button 8 is accessible through the disinfection device 1.

Hence, the disinfection device 1 of FIGS. 1 and 2 can be provided at a floor selection module of a pushing type, and light barriers or proximity sensors are installed inside the opening to detect fingers coming in or going out. The UVC-emitting semiconductor chip 2 may be turned on several seconds later, for example, 2 s to 3 s after the security sensor 4 detects whether the finger is completely out in order to make sure that no UVC directly irradiates to human fingers. The UVC will be turned on, for example, for 5 s to 10 s duration, which is enough to disinfect >99.9% under given distance with a single piece of a UVC LED which has in the range of 3 mW to 6 mW optical output, for example. For example, the indicator light source comprises one or a plurality of LEDs which may have any wavelength that is in the visible range of 380 nm to 780 nm, and the indicator LEDs are synchronized with the UVC LED operation, in order to indicate that the UVC LED is on or off.

In FIG. 3, a disinfection arrangement 10 comprising a disinfection device 1 and a button 6 is illustrated. The disinfection device 1 is in general configured like the disinfection device 1 of FIGS. 1 and 2, but additionally comprises an intermediate plate 54 located in the opening 56. The intermediate plate 54 is of a UVC-transparent material like acrylic glass, for example, polymethylmethacrylat, PMMA, or perfluoralkoxy, PFA. The intermediate plate 54 could be designed as a movable press wheel or chain, and a mechanical connection with the housing body 50.

For example, the intermediate plate 54 is connected with the top face of the cavern 52 by means of at least one first spring 91. Alternatively or additionally to the first spring 91, at a bottom of the intermediate plate 54 there can be at least one second spring 92 that rebounds the intermediate plate 54 from the button 6 after being pushed.

As an option, the intermediate plate 54 is composed of a couple of platelets 88 connected by joints 89. The joints 89 may be configured as springs, too. For example, there are at least two or at least three and/or at most ten of the platelets 88.

Because the intermediate plate 54 is transmissive for the UVC, a side of the intermediate plate 54 facing the top side 55 can be disinfected with the semiconductor chip 2 through the intermediate plate 54. Hence, the whole intermediate plate 54 can be disinfected due to transmissive behavior to UVC radiation. In this configuration it is possible that only a minor part of the UVC reaches the button 6 so that damage to the button 6 by the UVC can be reduced or avoided.

Moreover, in FIG. 3 it is illustrated that the fastener 11 may be based on screws so that the disinfection device 1 can be screwed on a board carrying the button 6. At a side of the carrier 90 facing the button 6, in this configuration there can be a non-skid support 94 or also the adhesive tape.

Otherwise, the same as to FIGS. 1 and 2 may also apply to FIG. 3, and vice versa.

According to FIG. 4, the intermediate plate 54 is not attached with springs to the top face of the cavern 52, but is a spring itself. Thus, the intermediate plate 54 may be fixed to the top face and rebounds itself back after the intermediate plate 54 and, thus, the button 6 is pressed by the user.

As a further option, there can be a slit 95 between a bottom part and a top part of the housing body 55. Hence, the top part may be reversibly removable from the bottom part, for example, to enable displacement of the batteries 93. In this case, for example, there can be the adhesive tape at the carrier 90 as well as the screws or clips accessible, for example, from the top side 55.

Otherwise, the same as to FIG. 3 may also apply to FIG. 4, and vice versa.

In the embodiment of FIGS. 5 and 6, the disinfection device 1 comprises a joint 59 like a tilt joint. The joint 59 connects a hand lever 96 and a button lever 97. The opening 56 to be grasped in by the user 7 is located at the hand lever 96. It is possible that the housing 5 completely covers the button 6 so that the button 6 itself can only indirectly be actuated by means of the disinfection device 1. When the hand lever 96 is no longer pulled by the user 7, then the hand lever 96 rebounds back to a standard upright position, for example.

Preferably, the semiconductor chip 2 is at a side of the housing facing the button 6 so that an inner side of the hand lever 96 can be disinfected. By means of a proximity sensor or a light barrier as the security sensor, not shown, the semiconductor chip 2 is turned on only when there is no user 7.

It is possible that the semiconductor chip 2 is located at a bottom of the opening 56 next to the joint 59. Thus, the hand lever 96 itself may be the radiation shield 3.

Hence, the disinfection device 1 can be arranged in a floor selection module of a pull switch type, and the pull switch type is designed for activating, for example, a floor selection by pulling the hand lever 96 instead of pushing it in a conventional way. Pulling the hand lever 96 will mechanically push and press the floor selection button 6. The indicator light sources 8 are synchronized with the UVC LED operation, in order to indicate that the UVC LED 2 is on or Off, and indicator light I is emitted accordingly.

For example, both the UVC and the indicator light I is turned on simultaneously 3 s after releasing the pull switch 96. No additional optical sensors like light barriers or proximity sensors might be required in this pull switch type, so that the security sensor may be a position sensor for the hand lever 96. UVC will be exposed only inside of the pull switch 96. In particular this kind of disinfection device 1 can be used not only for elevator floor selection but also for door openers in taxis or driving school cars or in public means of transports, and the like.

For example, the button 6 in particular of FIGS. 5 and 6 is an opener, possibly in a rental car, or is an airplane luggage door opener, or the like. Hence, the term ‘button’ can also include openers, door knobs, handgrips and the like.

Otherwise, the same as to FIGS. 1 to 4 may also apply to FIGS. 5 and 6, and vice versa.

In the embodiment of FIGS. 7 to 13, the disinfection arrangement 10 comprises a plurality of the buttons 6 arranged, for example, in two vertical rows. Each one of the buttons 6 is assigned a unit of the disinfection device 1, see the box in FIG. 8.

Atop the buttons 6, there are funnels 31 of the radiation shield 3. At a bottom of the funnels 31, as an option there is in each case a shutter 57. For example, the shutter 57 comprises two flaps. The funnels 31 and the shutters 57 are of UVC-opaque materials or carry a UVC-opaque coating. Such a coating, for example, made of zinc oxide, may be limited to an interior of the funnels 31 facing the cavern 52. By means of the shutter 57 and the funnel 31, the respective opening 56 is separated in each case in a forecourt 58 and a remaining part. However, the forecourts 58 and, thus, the funnels 31 are optional so that the shutters 57 may be located directly at the top side 55. The housing body 50 is of a UVC-opaque material, too, like a metal, for example, aluminum.

For example, the semiconductor chips 2 are located on two opposite vertical ends of the housing body 50. Again, there can be indicator light sources, not shown, synchronized with the UVC-emitting semiconductor chips 2. When the shutters 57 are closed, no UVC can leave the housing 5.

The shutters can be simply attached with springs to make it closed all the time except pushing it with fingers or even can be implemented with a shutter actuator 98 like a motor and some mechanical connection in order to open/close it automatically using signals from the security sensors 4.

By means of the security sensors 4, fingers can be sensed which are present inside the forecourts 58, and another sensor may detect the open or close state of the shutters 57. The shutter actuators 98 may each comprise a spring used for keeping the shutters 57 closed without any actions to them. The strength of such springs should be just as strong as maintain the shutters 57 closed, but also has to be lose enough to prevent hurting fingers when the motor 98 is not operating properly. However, in the intended use the shutters 57 themselves are configured not to be touched. For example, UVC generation may automatically be turned off if a finger grasps into the openings 56 when disinfection is running. Hence, by means of the sensors touching the shutters 57 can be inhibited.

For example, the shutters 57 are made of a material transparent in the visible spectral range like glass so that UVC is blocked but the buttons 6 can be seen. The cup-like funnels 31 may be tapered and/or may have a paraboloid shape, see FIG. 13.

For example, the UVC LEDs 2 turn on as stated in the following process scheme:
The sensor 4 senses the presence of the finger.
The respective shutter 57 opens.
The finger touches the respective floor selection button 6.
The finger comes completely out of the assigned opening 56.
The respective shutter 57 closes.
The UVC LEDs 2 turn on and automatically turn off after, for example, 30 s.

The disinfection device 1 of FIGS. 7 to 13 may be used for a single button 6 or for arrayed buttons 6; the same applies for all other exemplary embodiments.

Otherwise, the same as to FIGS. 1 to 6 may also apply to FIGS. 7 to 13, and vice versa.

The components shown in the figures follow, unless indicated otherwise, exemplarily in the specified sequence directly one on top of the other. Components which are not in contact in the figures are exemplarily spaced apart from one another. If lines are drawn parallel to one another, the corresponding surfaces may be oriented in parallel with one another. Likewise, unless indicated otherwise, the positions of the drawn components relative to one another are correctly reproduced in the figures.

The invention described here is not restricted by the description on the basis of the exemplary embodiments. Rather, the invention encompasses any new feature and also any combination of features, which includes in particular any combination of features in the patent claims, even if this feature or this combination itself is not explicitly specified in the patent claims or exemplary embodiments.

This patent application claims the priority of German patent application 10 2021 129 558.0, the disclosure content of which is hereby incorporated by reference.

### LIST OF REFERENCE SIGNS

1 disinfection device
2 UVC-emitting semiconductor chip
3 radiation shield
31 funnel
4 security sensor
5 housing
50 housing body
51 reflection layer for the ultraviolet C radiation
52 cavern
53 absorption layer for the ultraviolet C radiation
54 intermediate plate
55 top side
56 opening
57 shutter
58 forecourt
59 tilt joint
6 button
7 user
8 indicator light source
88 platelet
89 joint
90 carrier
91 first spring
92 second spring
93 battery
94 non-skid support
95 slit
96 hand lever
97 button lever
98 shutter actuator
10 disinfection arrangement
11 fastener
I indicator light
UVC ultraviolet C radiation

1. A disinfection device comprising:
a housing comprising a housing body,
a semiconductor chip located in the housing body and configured to emit ultraviolet C radiation (UVC), and
a security sensor located in the housing body, wherein
the housing body forms a radiation shield preventing the ultraviolet C radiation (UVC) from leaving the disinfection device,
the housing body comprises an opening configured for actuating a button by a user, to actuate the button the user has to grasp into the opening (56),
the semiconductor chip is configured to irradiate at least part of the opening and/or the button, and
the disinfection device is configured as an attachment device to be disposed over the button,
and wherein one of the following possibilities holds true:
the housing further comprises an intermediate plate located movably in the opening between a top side of the housing body and the button, the intermediate plate is configured to press the button when being actuated by the user, or
the housing further comprises a shutter forming part of the radiation shield, the shutter closes the opening when being closed and allows for accessing the button when being opened, the security sensor is configured to provide an opening signal for the shutter when the semiconductor chip is turned off and when the user approaches the shutter.

2. The disinfection device according to the preceding claim 1,
wherein the opening runs completely through the housing body and is, seen in top view of the housing body, completely surrounded by the housing body,
wherein the opening is configured to freely access the button by the user.

3. The disinfection device according to claim 1,
wherein the semiconductor chip is located in a cavern within the housing body, the cavern is formed around the opening, seen in top view of the housing body, and is in contact with the opening,
wherein a top face of the cavern is coated with a reflection layer for the ultraviolet C radiation (UVC) of the radiation shield, and side faces of the opening are coated with an absorption layer for the ultraviolet C radiation (UVC).

4. The disinfection device according to claim 1,
wherein the housing further comprises the intermediate plate located movably in the opening between the top side of the housing body and the button,
and the intermediate plate is configured to press the button when being actuated by the user.

5. The disinfection device according to the preceding claim 1,
wherein the intermediate plate is at least partially transparent for the ultraviolet C radiation (UVC),
wherein the intermediate plate is located between the top side and the semiconductor chip.

6. The disinfection device according to claim 1,
wherein the housing further comprises the shutter forming part of the radiation shield, the shutter closes the opening when being closed and allows for accessing the button when being opened,
and the security sensor is configured to provide the opening signal for the shutter when the semiconductor chip is turned off and when the user approaches the shutter.

7. The disinfection device according to claim 6,
wherein the shutter is located at a bottom of a forecourt of the opening, the forecourt is surrounded all around by a funnel of the radiation shield in a lateral direction.

8. The disinfection device according to claim 1,
wherein the housing further comprises a joint,
wherein a first end at the tilt is configured to be pulled by the user and a second end at the joint is configured to push the button.

9. The disinfection device according to the preceding claim 8,
wherein the semiconductor chip is configured to irradiate an interior side of the second end, wherein the opening is located between the semiconductor chip and the second end.

10. The disinfection device according to claim 1,
further comprising an indicator light source configured to indicate when the semiconductor chip is turned on,
wherein the indicator light source is embedded in the housing body and the housing body is of a material transparent for visible light.

11. The disinfection device according to claim 1,
further comprising a battery, the disinfection device is configured to be powered by the battery.

12. The disinfection device according to claim 1,
wherein a distance between a surface to be disinfected by the ultraviolet C radiation (UVC) and the semiconductor chip is at most 4 cm.

13. The disinfection device according to claim 1, any one of the preceding claims,
wherein the security sensor comprises a light barrier that is configured to check an entrance of the opening,
wherein the semiconductor chip is a light-emitting diode chip or a laser diode chip.

14. The disinfection device according to claim 1,
wherein the button is a pushbutton, a pressure key, a switch, a shifter, an opener, a knob, a lever, a touchpad, a shutter, a cover film or a handgrip which is configured to be touched by a user.

15. A disinfection arrangement comprising a disinfection device according to claim 1 of the preceding claims and a button,
wherein the disinfection device is attached over the button so that the button can be actuated only by means of grasping, by the user, into the opening.

16. The disinfection arrangement according to claim 15,
wherein the disinfection device comprises a fastener, and the disinfection device is attached over the button by means of the fastener,
wherein the fastener comprises an adhesive tape.

17. An operation method for a disinfection device according to claim 1,
wherein during operation the semiconductor chip has an optical output of the ultraviolet C radiation (UVC) of at least 1 mW and of at most 20 mW,
wherein for disinfection the semiconductor chip is turned on for at least 1 s and for at most 20 s.
